(19) United States
(12) Patent Application Publication
(10) Pub. No.: US 2018/0369083 A1
(43) Pub. Date: Dec. 27, 2018

(54) WATER-IN-OIL EMULSION WITH MOISTURIZING EFFECT CONTAINING HYDROPHOBIC COATED PIGMENTS AND AN AQUEOUS PHASE AT HIGH CONTENT

(71) Applicant: L'OREAL, Paris (FR)

(72) Inventor: Elodie VALVERDE, Chevilly La Rue (FR)

(73) Assignee: L'OREAL, Paris (FR)

(21) Appl. No.: 16/062,422

(22) PCT Filed: Dec. 7, 2016
(86) PCT No.: PCT/EP2016/080108

§ 371 Date: Jun. 14, 2018

(30) Foreign Application Priority Data
Dec. 17, 2015 (FR) 1562608
Dec. 17, 2015 (FR) 1562610

(51) Int. Cl. A61K 8/06; A61K 8/894; A61K 8/893; A61K 8/368; A61K 8/37; A61K 8/58; A61K 8/29; A61K 8/34; A61Q 1/02; A61Q 19/00

(52) U.S. Cl. CPC A61K 8/064; A61K 8/894; A61K 8/893; A61K 8/368; A61K 8/37; A61K 8/585; A61K 2800/651; A61K 8/345; A61Q 1/02; A61Q 19/00; A61K 2800/43; A61K 2800/622; A61K 8/29

(57) ABSTRACT

The present invention relates to a in the form of an emulsion, in particular a water-in-oil emulsion, in particular comprising a physiologically acceptable medium, in particular for coating keratin materials, more particularly for making up and/or caring for keratin materials, such as the skin, containing: —at least one aqueous phase in a content from 60% to 80% by weight relative to the total weight of the composition; and —at least one oily phase at a concentration of less than 30.0% by weight relative to the total weight of the composition and comprising at least one silicone oil; and —at least one hydrophobic coated pigment; and —optionally at least one moisturizing agent at a concentration ranging from 10% to 25% by weight relative to the total weight of the composition; and —at least one emulsifying surfactant. The invention also relates to a process for coating keratin materials, more particularly for making up and/or caring for keratin materials, such as the skin, characterized in that it comprises the application to the keratin materials of a composition as defined previously.

The present invention is directed towards proposing for the field of caring for and/or making up keratin materials, in particular the skin and/or the lips, and in particular the skin, and keratin fibres, in particular the eyebrows, a novel emulsion form that is most particularly advantageous with regard to its technical performance and the sensations it affords the user during its application thereto, in particular to the skin.

Cosmetic compositions, for example foundations, are commonly used to give the skin an aesthetic colour, but also to enhance the beauty of irregular skin, by making it possible to hide marks and dyschromias, to reduce the visibility of relief imperfections such as pores and wrinkles, and to conceal spots and acne marks; in this regard, coverage is one of the main properties sought.

Emulsions are generally attractive, in particular in the context of foundations, for consumers since they are easy to apply. Considerable coverage is often sought for such compositions, in order to mask skin defects and to make the complexion uniform. Such coverage is often obtained using a high proportion of pigments, which results in a very matt and unnatural result. However, the emulsions generally used in makeup have a tendency to produce on the skin during application are not very effective in terms of freshness and moisturization. It is also important for the pigments present in these compositions to have good dispersibility with a view to obtaining a stable and uniform composition. Hydrophobic coated pigments are particularly advantageous in particular in foundations because they provide better adhesion and better wear and also have a better cosmeticity on application.

Compositions with a moisturizing effect for skin care, in the form of emulsions comprising an emulsifying silicone elastomer, and an oily phase comprising a silicone oil, are known in documents US 20130345317, US2015250706, EP 2 264 000 and US2015017047. These formulations have not been proposed for making up keratin materials or for containing hydrophobic coated pigments.

There remains the need to find novel emulsions based on hydrophobic coated pigments, which are stable and uniform, and which confer good sensory properties in terms of freshness and a good moisturizing effect while at the same time having good makeup properties, such as good coverage and good uniformity of the complexion.

There also remains the need to find novel emulsions based on hydrophobic coated pigments, which are stable and uniform, and which confer good makeup properties such as good coverage, a good shininess giving the complexion a natural radiance and also good sensory properties such as freshness, lightness on application, and a non-tacky effect without the drawbacks mentioned above.

The applicant has discovered, surprisingly, that this objective can be achieved with a composition in the form of an emulsion, in particular a water-in-oil emulsion, in particular comprising a physiologically acceptable medium, in particular for coating keratin materials, more particularly for making up and/or caring for keratin materials, such as the skin, containing:
at least one aqueous phase in a content of 60% to 80% by weight relative to the total weight of the composition; and
at least one oily phase at a concentration of less than 30.0% by weight relative to the total weight of the composition and comprising at least one silicone oil; and
at least one hydrophobic coated pigment; and
optionally at least one moisturizing agent at a concentration ranging from 10% to 25% by weight relative to the total weight of the composition;
at least one emulsifying surfactant, preferably chosen from non-ionic emulsifying surfactants, and more particularly emulsifying silicone elastomers.

More particularly, the composition has a viscosity at 25° C. ranging from 0.1 to 10 Pa·s.

This discovery forms the basis of the invention.

Thus, according to one of its aspects, the present invention relates to a composition in the form of an emulsion, in particular a water-in-oil emulsion, in particular comprising a physiologically acceptable medium, in particular for coating keratin materials, more particularly for making up and/or caring for keratin materials, such as the skin, containing:
at least one aqueous phase in a content of 60% to 80% by weight relative to the total weight of the composition; and
at least one oily phase at a concentration of less than 30.0% by weight relative to the total weight of the composition and comprising at least one silicone oil; and
at least one hydrophobic coated pigment; and
optionally at least one moisturizing agent at a concentration ranging from 10% to 25% by weight relative to the total weight of the composition;
at least one emulsifying surfactant, preferably chosen from non-ionic emulsifying surfactants, and more particularly emulsifying silicone elastomers.

According to one particular mode of the invention, the composition is in the form of an emulsion, in particular comprising a physiologically acceptable medium, in particular for coating keratin materials, more particularly for making up and/or caring for keratin materials, such as the skin, containing:
at least one aqueous phase in a content ranging from 60% to 80% by weight relative to the total weight of the composition; and
at least one oily phase at a concentration of less than 30.0% by weight relative to the total weight of the composition, and comprising at least one silicone oil; and
at least one hydrophobic coated pigment; and
at least one emulsifying silicone elastomer.

According to another particular mode of the invention, the composition is in the form of a water-in-oil emulsion containing:
at least one continuous oily phase; and
at least one aqueous phase in a content ranging from 60% to 80% by weight relative to the total weight of the composition; and
at least one oily phase at a concentration of less than 30.0% by weight relative to the total weight of the composition and comprising at least one silicone oil; and
at least one hydrophobic coated pigment; and
at least one moisturizing agent at a concentration ranging from 10% to 25% by weight relative to the total weight of the composition,
at least one emulsifying surfactant, preferably chosen from non-ionic emulsifying surfactants, and more particularly emulsifying silicone elastomers.
   said composition having a viscosity at 25° C. ranging from 0.1 to 10 Pa·s, preferably from 0.5 to 5 Pa·s and more preferentially from 1.5 to 3 Pa·s.

The viscosity of the composition is measured at 25° C. using a Rheomat RM100 Touch® (from the company Lamy), equipped with an MS-R3 spindle rotating at a rotational speed of 200 revolutions/minute. The measurement is taken after 10 minutes of rotation. The viscosity measurements are taken not more than 1 week after production.

The invention also relates to a process for coating keratin materials, more particularly for making up and/or caring for keratin materials, such as the skin, characterized in that it comprises the application to the keratin materials of a composition such as one of those defined above.

### Definitions

The term “physiologically acceptable” is intended to mean compatible with the skin and/or its superficial body growths, which exhibits a pleasant colour, odour and feel and which does not generate unacceptable discomfort (tingling, tautness) liable to dissuade the consumer from using this composition.

For the purposes of the present invention, the term “emulsion” is intended to mean any composition comprising at least two phases that are liquid at ambient temperature (20-25° C.) and immiscible with respect to one another, one of the two phases being dispersed in the other phase in the form of droplets so as to observe a mixture that is macroscopically uniform to the naked eye; in particular, said composition in the form of a water-in-oil emulsion, also called inverse emulsion, is constituted of a continuous oily phase in which the aqueous phase is dispersed in the form of droplets so as to observe a mixture that is macroscopically uniform to the naked eye.

In the context of the present invention, the term “keratin material” is intended to mean, in particular, the skin (body, face, area around the eyes), the lips, the eyelashes and the eyebrows. More particularly, the term “keratin material” is intended to mean the skin.

The term “physiologically acceptable” is intended to mean compatible with the skin and/or its superficial body growths, which exhibits a pleasant colour, odour and feel and which does not generate unacceptable discomfort (tingling, tautness) liable to dissuade the consumer from using this composition.

### Preparation Process

According to one particular mode, the water-in-oil emulsions comprising:
at least one aqueous phase in a content ranging from 60% to 80% by weight relative to the total weight of the composition; and
at least one oily phase in a content of less than 30.0% by weight relative to the total weight of the composition and comprising at least one silicone oil; and
at least one hydrophobic coated pigment; and
optionally at least one moisturizing agent at a concentration ranging from 10% to 25% by weight relative to the total weight of the composition; and
at least one emulsifying surfactant, are obtained by means of a preparation process comprising the following steps:
   a) the aqueous phase is prepared by mixing the water, the optional moisturizing agent(s) and the optional other ingredients, with magnetic stirring, at a temperature of from 50 to 60° C. from 3 to 10 minutes and is then left to cool to 25-30° C.;
   b) the oily phase is prepared, at a temperature ranging from 25 to 30° C., by mixing the hydrophobic coated pigment, the surfactant emulsifier(s) and the oily component(s) with rotor stator stirring (Moritz) for 15 to 25 minutes at a shear rate ranging from 2500 to 3500 revolutions/minute; then
   c) the oily phase is stirred in a Rayneri deflocculating paddle mixer at a shear rate ranging from 300 to 500 revolutions/minute, at a temperature of from 25 to 30° C.; then
   d) the aqueous phase is added to said oily phase, over the course of a period of time not exceeding 1 minute, while increasing the stirring of the Rayneri mixer to a shear rate ranging from 700 to 1200 revolutions/minute, preferably up to 1000 revolutions/minute, at a temperature of from 25 to 30° C.;
   e) the resulting mixture is left to stir for a further period of time ranging from 1 to 3 minutes, at a shear rate ranging from 700 to 1200 revolutions/minute, at a temperature ranging from 25 to 30° C.

According to one preferred mode of the preparation process, pre-milling of the hydrophobic coated pigments is carried out. The pigments can in particular be milled in oil using a three-roll mill with 3 openings (large, medium then small).

The present invention also relates to a water-in-oil emulsion that can be obtained by means of this preparation process.

### Aqueous Phase

The aqueous phase of a composition according to the invention comprises water and optionally any water-soluble or water-miscible ingredient, such as water-soluble solvents.

A water that is suitable for use in the invention may be a floral water such as cornflower water and/or a mineral water such as Vittel water, Lucas water or La Roche Posay water and/or a spring water.

In the present invention, the term “water-soluble solvent” denotes a compound that is liquid at ambient temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made in particular of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

The aqueous phase is present in a concentration ranging from 60% to 80% by weight, more particularly from 65% to 75% by weight, relative to the total weight of said composition.

According to one particular form of the invention, the aqueous phase of the emulsion is characterized by a number of droplets of less than 200 000, preferably less than 150 000, more preferentially less than 100 000.

The measurement of the number of droplets of the aqueous phase per mm² of cross section of composition is carried out at 20° C. using an SP8 laser scanning confocal microscope (LSCM) (Leica Microsystems, Germany). The objective used for the observation is an HC PL APO CS2×40 oil-immersion objective with an index n=1.518 and a digital aperture of 1.3. A drop of oil is deposited on the objective before placing the coverslip on the sample holder.

### Fluorescent Labelling of the Phases of Interest:

1 g of sample is taken from the mass and is placed in a 5 ml vial. 10 μl of an aqueous solution of fluorescein (Fluorescein Sodium, Merck), having a concentration of 10⁻¹ M, are added using a micropipette. This mixture is gently homogenized by hand using a spatula for 30 seconds. The fluorescein solution defuses by affinity in the water droplets dispersed in the emulsion.

### Preparation of the Coverslip:

A small amount of emulsion (˜200 mg) labelled with the aqueous fluorescein solution is deposited on a #1 microscope coverslip, 24×60 mm in size, approximately 170 μm thick (Menzel-Gläser).

### Observation:

A scan speed of 600 Hz is defined for a logical image size of 512×512 pixels. Using a ×5 digital zoom, the magnification of the final image is equivalent to ×200, which corresponds to a physical size of the image equal to 58.12 μm×58.12 μm or else a surface area of 3377.93 μm².

The fluorescein is excited using an Argon 488 nm laser, power set at 0.0520 W, beam intensity at 5%. The position of the upper face of the coverslip (z0) is determined by detection of a maximum intensity emitted following the reflection of the laser on the coverslip. On the basis of this position z0, the focal plane of observation is defined at z=z0+15 μm. This position makes it possible to dispense with the potential artifacts of interactions between the glass and the sample.

The emission of fluorescence is detected using a photomultiplier positioned on the wavelength range 505-540 nm, with a sufficient gain so as not to saturate the pixels making it possible to reconstitute the image.

### Determination of the Number of Droplets:

The number of droplets is counted on an observation area of 50×50 μm, then the figure obtained is multiplied by 400. The value of the number of droplets of the aqueous phase per mm² of cross-section of composition is then obtained.

### Oily Phase

The emulsion of the invention also comprises an oily phase. Said phase is liquid (in the absence of structuring agent) at ambient temperature (20-25° C.). Preferentially, the water-immiscible organic liquid phase in accordance with the invention generally comprises at least one volatile oil and/or one non-volatile oil and optionally any ingredient that is soluble or miscible in the oily phase.

The term “oil” is intended to mean a fatty substance that is liquid at ambient temperature (25° C.) and atmospheric pressure (760 mmHg, i.e. 105 Pa). The oil may be volatile or non-volatile.

For the purposes of the invention, the term “volatile oil” is intended to mean an oil that is capable of evaporating on contact with the skin or the keratin fibre in less than one hour, at ambient temperature and atmospheric pressure. The volatile oils of the invention are volatile cosmetic oils, which are liquid at ambient temperature, having a non-zero vapour pressure, at ambient temperature and atmospheric pressure, ranging in particular from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” is intended to mean an oil that remains on the skin or the keratin fibre at ambient temperature and atmospheric pressure for at least several hours, and that in particular has a vapour pressure of less than 10⁻³ mmHg (0.13 Pa).

The oil may be chosen from any oil, which are preferably physiologically acceptable oils, in particular mineral, animal, plant or synthetic oils; in particular volatile or non-volatile hydrocarbon-based oils and/or silicone oils and/or fluorinated oils, and mixtures thereof.

More specifically, the term “hydrocarbon-based oil” is intended to mean an oil mainly comprising carbon and hydrogen atoms and optionally one or more functions chosen from hydroxyl, ester, ether and carboxylic functions. Generally, the oil has a viscosity of from 0.5 to 100 000 mPa·s, preferably from 50 to 50 000 mPa·s and more preferably from 100 to 300 000 mPa·s.

For the purposes of the present invention, the term “silicone oil” is intended to mean an oil comprising at least one silicon atom, and in particular at least one Si—O group.

For the purpose of the present invention, the term “fluoro oil” is intended to mean an oil comprising at least one fluorine atom.

The oily phase concentration of the emulsion of the invention is less than 30.0% by weight, more preferentially less than or equal to 25.0% by weight and even better still ranges from 10% to 25% by weight and more particularly ranges from 15% to 20% by weight relative to the total weight of the composition.

As examples of a volatile hydrocarbon-based oil that can be used in the invention, mention may be made of:
hydrocarbon-based oils containing from 8 to 16 carbon atoms, and in particular C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C₈-C₁₆ esters and isohexyl neopentanoate, and mixtures thereof. Use may also be made of other volatile hydrocarbon-based oils, such as petroleum distillates, in particular those sold under the name Shell Solt by Shell; volatile linear alkanes, such as those described in Patent Application DE10 2008 012 457 from Cognis.

By way of example of a volatile silicon oil that can be used in the invention, mention may be made of volatile silicone oils, for instance linear or cyclic volatile silicone oils, in particular those having a viscosity≤8 centistokes (8×10⁻⁶ m²/s), and containing in particular from 2 to 7 silicon atoms, these silicones optionally comprising alkyl or alkoxy groups containing from 1 to 10 carbon atoms. As volatile silicone oils that may be used in the invention, mention may be made in particular of caprylyl methicone, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane;
and mixtures thereof.

As examples of non-volatile hydrocarbon-based oils that may be used in the invention, mention may be made of:
hydrocarbon-based oils of animal origin, such as perhydrosqualene;
linear or branched hydrocarbons of mineral or synthetic origin, such as liquid paraffins and their derivatives, petrolatum, polydecenes, polybutenes, hydrogenated polyisobutene, such as Parleam, or squalane;
phytostearyl esters, such as phytostearyl oleate, phytostearyl isostearate and lauroyl/octyldodecyl/phytostearyl glutamate (Ajinomoto, Eldew PS203®),
triglycerides constituted of fatty acid esters of glycerol, in particular the fatty acids of which may have chain lengths ranging from C₄ to C₃₆, and in particular from C₁₈ to C₃₆, these oils possibly being linear or branched, and saturated or unsaturated; these oils may in particular be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea oil; or alternatively caprylic/capric acid triglycerides, for instance those sold by the company Stéarineries Dubois or those sold under the names Miglyol 810®, 812® and 818® by the company Dynamit Nobel;
synthetic ethers containing from 10 to 40 carbon atoms, such as dicaprylyl ether,
hydrocarbon-based esters of formula RCOOR′ in which RCOO represents a carboxylic acid residue comprising from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and in particular isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethyl hexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate;
polyesters obtained by condensation of an unsaturated fatty acid dimer and/or trimer and of diol, such as those described in patent application FR 0 853 634, in particular such as dilinoleic acid and 1,4-butanediol. Mention may in particular be made in this respect of the polymer sold by Biosynthis under the name Viscoplast 14436H® (INCI name: Dilinoleic Acid/Butanediol Copolymer), or copolymers of polyols and of diacid dimers, and esters thereof, such as Hailuscent ISDA®,
polyol esters and pentaerythritol esters, for instance dipentaerythritol tetrahydroxystearate/tetraisostearate,
fatty alcohols containing from 12 to 26 carbon atoms, for instance octyldodecanol, 2-butyloctanol, 2-hexyldecanol, 2-undecylpentadecanol and oleyl alcohol,
dialkyl carbonates, the two alkyl chains possibly being identical or different, such as dicaprylyl carbonate sold under the name Cetiol CC® by Cognis, and
vinylpyrrolidone copolymers such as the vinylpyrrolidone/1-hexadecene copolymer, Antaron V-216 sold or manufactured by the company ISP,
linear fatty acid esters with a total carbon number ranging from 35 to 70, such as pentaerythrityl tetrapelargonate,
hydroxylated esters such as polyglyceryl-2 triisostearate,
aromatic esters such as tridecyl trimellitate, C₁₂-C₁₅ alcohol benzoate, the 2-phenylethyl ester of benzoic acid, and butyloctyl salicylate,
C₂₄-C₂₈ esters of branched fatty alcohols or fatty acids such as those described in patent application EP-A-0 955 039, and in particular triisoarachidyl citrate, pentaerythrityl tetra isononanoate, glyceryl triisostearate, glyceryl tris(2-decyl)tetradecanoate, pentaerythrityl tetra isostearate, polyglyceryl-2 tetra isostearate or pentaerythrityl tetrakis(2-decyl)tetradecanoate,
esters and polyesters of dimer diol and of monocarboxylic or dicarboxylic acid, such as esters of dimer diol and of fatty acid and esters of dimer diol and of dimer dicarboxylic acid, such as Lusplan DD-DA5® and Lusplan DD-DA7® sold by the company Nippon Fine Chemical and described in patent application US 2004-175 338, the content of which is incorporated into the present application by reference,
and mixtures thereof.

According to one particular mode, the non-volatile hydrocarbon-based oil may be chosen from liquid lipophilic organic UV-screening agents.

The term “liquid lipophilic organic UV-screening agent” is intended to mean any organic chemical molecule capable of absorbing at least UV radiation in the wavelength range of between 280 and 400 nm, said molecule being in liquid form at ambient temperature (20-25° C.) and at atmospheric pressure (760 mmHg) and capable of being miscible in an oily phase.

The liquid organic UV-screening agents that can be used according to the invention may be chosen from
liquid lipophilic β,β-diphenylacrylate compounds,
liquid lipophilic salicylate compounds,
liquid lipophilic cinnamate compounds,
and mixtures thereof.

### i) β,β-Diphenylacrylate Compounds

Among the liquid lipophilic organic UVB-screening agents that can be used according to the invention, mention may be made of the liquid lipophilic alkyl β,β-diphenylacrylate or α-cyano-β,β-diphenylacrylate compounds of formula (I) below:

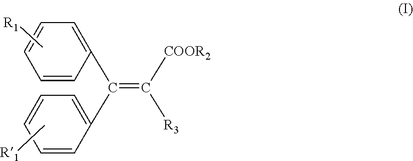

where R₁ to R₃ can have the following meanings:
R₁ and R′₁, which may be identical or different, represent a hydrogen atom, a straight-chain or branched-chain C₁-C₈ alkoxy radical or a straight-chain or branched-chain C₁-C₄ alkyl radical;
R₁ and R′₁ being in the para meta position;
R₂ represents a straight-chain or branched-chain C₁-C₁₂ alkyl radical;
R₃ represents a hydrogen atom or the CN radical.

Among the straight-chain or branched-chain C₁-C₈ alkoxy radicals, mention may for example be made of methoxy, ethoxy, n-propoxy, isopropoxy, n-butoxy, isobutoxy, tert-butoxy, n-amyloxy, isoamyloxy, neopentyloxy, n-hexyloxy, n-heptyloxy, n-octyloxy and 2-ethylhexyloxy radicals.

Among the straight-chain or branched-chain C₁-C₄ alkyl radicals, mention may more particularly be made of methyl, ethyl, n-propyl, isopropyl, n-butyl, isobutyl and tert-butyl radicals. For the C₁-C₁₂ alkyl radicals, mention may be made, by way of example, in addition to those mentioned above, of n-amyl, isoamyl, neopentyl, n-hexyl, n-heptyl, n-octyl, 2-ethylhexyl, decyl and lauryl radicals. Among the compounds of general formula (I), the following compounds are more particularly preferred:
2-ethylhexyl α-cyano-β,β-diphenylacrylate or Octocrylene, sold in particular under the trade name Uvinul N539® by BASF;
ethyl α-cyano-β,β-diphenylacrylate such as Etocrylene, sold in particular under the trade name Uvinul N35® by BASF;
2-ethylhexyl β,β-diphenylacrylate;
ethyl β,β-di(4′-methoxyphenyl)acrylate.

Among the compounds of general formula (I), the compound 2-ethylhexyl 2-cyano-3,3-diphenylacrylate or Octocrylene is even more particularly preferred.

### ii) Salicylate Compounds

Among the liquid lipophilic salicylate compounds that can be used according to the invention, mention may be made of:
Homosalate, sold under the name Eusolex HMS® by Rona/EM Industries,
Ethylhexyl salicylate, sold under the name Neo Heliopan OS® by Symrise.
   iii) Cinnamate Compounds

Among the liquid lipophilic cinnamate compounds that can be used according to the invention, mention may be made of:
Ethylhexyl Methoxycinnamate, sold in particular under the trade name Parsol MCX® by DSM Nutritional Products,
Isopropyl Methoxycinnamate,
Isoamyl Methoxycinnamate, sold under the trade name Neo Heliopan E 1000 by Symrise.

Among the liquid lipophilic screening agents according to the invention, use will more particularly be made of the compound Ethylhexyl Methoxycinnamate.

Among the non-volatile fluorinated and/or silicone oils, mention may be made of:
fluorinated oils which are optionally partially hydrocarbon-based and/or silicone-based, for instance fluorosilicone oils, fluorinated polyethers or fluorinated silicones, such as described in the document EP-A-847 752;
silicone oils, such as non-volatile polydimethylsiloxanes (PDMSs); phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Preferentially, the oily phase comprises at least one silicone oil, even more preferentially chosen from:
volatile cyclic silicone oils having a viscosity at ambient temperature of less than 8 cst and containing in particular from 4 to 7 silicone atoms, these silicones optionally comprising alkyl or alkoxy groups containing from 1 to 10 carbon atoms, in particular chosen from hexamethylcyclotrisiloxane, octamethylcyclotetrasiloxane, decamethylcyclopentasiloxane and dodecamethylcyclohexasiloxane (cyclohexasiloxane), and mixtures thereof;
volatile or non-volatile polydimethylsiloxanes (PDMSs) (INCI name: Dimethicone);
phenylated silicones;
polydimethylsiloxanes comprising aliphatic groups, in particular alkyl groups, or alkoxy groups, which are pendent and/or at the end of the silicone chain; these groups each comprising from 6 and 24 carbon atoms, and more particularly caprylyl methicone, such as the commercial product Dow Corning FZ-3196® from the company Dow Corning;
mixtures thereof.

Preferentially, the silicone oil is caprylyl methicone.

According to one particular mode of the invention, the oily phase comprises at least one silicone oil as defined above and at least one liquid lipophilic organic UV-screening agent as defined above.

According to one particular mode of the invention, the oily phase comprises at least ethylhexyl methoxycinnamate and caprylyl methicone.

### Emulsifiers

The emulsions according to the invention generally comprise one or more emulsifying surfactants, which are preferably non-ionic.

For the purposes of the present invention, the term “emulsifying surfactant” is intended to mean an amphiphilic surfactant compound, i.e. one which has two parts of different polarity. Generally, one is lipophilic (soluble or dispersible in an oily phase). The other is hydrophilic (soluble or dispersible in water). The emulsifying surfactants are characterized by the value of their HLB (Hydrophilic Lipophilic Balance), the HLB being the ratio between the hydrophilic part and the lipophilic part in the molecule. The term “HLB” is well known to those skilled in the art and is described, for example, in “The HLB system. A time-saving guide to Emulsifier Selection” (published by ICI Americas Inc., 1984). For the emulsifying surfactants, the HLB generally ranges from 3 to 8 for the preparation of W/O emulsions. The HLB of the surfactant(s) used according to the invention may be determined via the Griffin method or the Davies method.

Examples of W/O emulsifying surfactants that may be mentioned include alkyl esters or ethers of sorbitan, of glycerol, of polyol or of sugars; silicone surfactants, for instance dimethicone copolyols, such as the one having the INCI name Dimethicone (and) PEG/PPG-18/18 Dimethicone sold under the brand X-22-6711D® by the company Shin Etsu, the mixture of cyclomethicone and of dimethicone copolyol, sold under the name DC 5225 C® by the company Dow Corning, and alkyldimethicone copolyols such as laurylmethicone copolyol sold under the name Dow Corning 5200 Formulation Aid by the company Dow Corning; cetyl dimethicone copolyol, for instance cetyl PEG/PPG-10/1 dimethicone, such as the product sold under the name Abil EM 90® by the company Evonik Goldschmidt, and the mixture of cetyl dimethicone copolyol, of polyglyceryl isostearate (4 mol) and of hexyl laurate, sold under the name Abil WE O9® by the company Goldschmidt. One or more coemulsifiers, which may be chosen advantageously from the group comprising polyol alkyl esters, may also be added thereto.

Mention may also be made of non-silicone emulsifying surfactants, in particular alkyl esters or ethers of sorbitan, of glycerol, of polyol or of sugars.

Polyol alkyl esters that may in particular be mentioned include polyethylene glycol esters, for instance PEG-30 dipolyhydroxystearate, such as the product sold under the name Arlacel P135® by the company ICI.

Examples of glycerol and/or sorbitan esters that may be mentioned include polyglyceryl isostearate (INCI name: Polyglyceryl-4 Isostearate, such as the product sold under the name Isolan GI 34® by the company Evonik Goldschmidt; sorbitan isostearate, such as the product sold under the name Arlacel 987® by the company ICI; sorbitan glyceryl isostearate, such as the product sold under the name Arlacel 986® by the company ICI, and mixtures thereof.

According to one particular form of the invention, the emulsifying surfactant can be chosen from emulsifying silicone elastomers.

The term “silicone elastomer” is intended to mean a supple, deformable organopolysiloxane that has viscoelastic properties and in particular the consistency of a sponge or a supple sphere. Its modulus of elasticity is such that this material withstands deformation and has a limited ability to extend and to contract. This material is capable of regaining its original shape after stretching.

The emulsifying silicone elastomer may be chosen from polyoxyalkylenated silicone elastomers and polyglycerolated silicone elastomers, and mixtures thereof.

a) Polyoxyalkylenated Silicone Elastomers

The polyoxyalkylenated silicone elastomer is a crosslinked organopolysiloxane that may be obtained by a crosslinking addition reaction of diorganopolysiloxane containing at least one hydrogen bonded to silicon and of a polyoxyalkylene containing at least two ethylenically unsaturated groups.

Preferably, the polyoxyalkylenated crosslinked organopolysiloxane is obtained by a crosslinking addition reaction (A1) of diorganopolysiloxane containing at least two hydrogens each bonded to a silicon, and (B1) of polyoxyalkylene containing at least two ethylenically unsaturated groups, in particular in the presence (C1) of a platinum catalyst, as described, for example, in patents U.S. Pat. No. 5,236,986 and U.S. Pat. No. 5,412,004.

In particular, the organopolysiloxane may be obtained by reaction of dimethylvinylsiloxy-terminated polyoxyalkylene (in particular polyoxyethylene and/or polyoxypropylene) and of trimethylsiloxy-terminated methylhydropolysiloxane, in the presence of a platinum catalyst.

The organic groups bonded to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A1) may thus be chosen from trimethylsiloxy-terminated methylhydropolysiloxanes, trimethylsiloxy-terminated dimethylsiloxane/methylhydrosiloxane copolymers, dimethylsiloxane/methylhydrosiloxane cyclic copolymers, and trimethylsiloxy-terminated dimethylsiloxane/methylhydrosiloxane/laurylmethylsiloxane copolymers.

Compound (C1) is the catalyst for the crosslinking reaction, and is in particular chloroplatinic acid, chloroplatinic acid-olefin complexes, chloroplatinic acid-alkenylsiloxane complexes, chloroplatinic acid-diketone complexes, platinum black and platinum on a support.

Advantageously, the polyoxyalkylenated silicone elastomers may be formed from divinyl compounds, in particular polyoxyalkylenes containing at least two vinyl groups, reacting with Si—H bonds of a polysiloxane.

The polyoxyalkylenated silicone elastomer according to the invention is preferably mixed with at least one hydrocarbon-based oil and/or one silicone oil to form a gel. In these gels, the polyoxyalkylenated elastomer can be in the form of non-spherical particles.

Polyoxyalkylenated elastomers are described in particular in U.S. Pat. No. 5,236,986, U.S. Pat. No. 5,412,004, U.S. Pat. No. 5,837,793 and U.S. Pat. No. 5,811,487.

As polyoxyalkylenated silicone elastomers, use may be made of those having the following INCI names:

### Dimethicone/PEG-10/15 Crosspolymer,

### PEG-15/Lauryl Dimethicone Crosspolymer,

### PEG-10/Lauryl Dimethicone Crosspolymer,

### PEG-12 Dimethicone Crosspolymer,

### PEG-10 Dimethicone Crosspolymer,

### PEG-10 Dimethicone/Vinyl Dimethicone Crosspolymer,

### PEG-12 Dimethicone/PPG-20 Crosspolymer,

and mixtures thereof.

They are in particular sold under the KSG® name by the company Shin Etsu

KSG-210® (INCI name: Dimethicone and Dimethicone/PEG-10/15-Crosspolymer;
KSG-310® (INCI name: PEG-15/Lauryl Dimethicone Crosspolymer and Mineral oil;
KSG-320® (INCI name: PEG-15/Lauryl Dimethicone Crosspolymer and Isododecane;
KSG-330® (INCI name: PEG-15/Lauryl Dimethicone Crosspolymer and Triethylhexanoin;
KSG-340® (INCI name: Squalane and PEG-15/Lauryl Dimethicone Crosspolymer.

They are in particular sold by the company Dow Corning under the name Dow Corning 9011 Silicone Elastomer Blend®; INCI name: Cyclopentasiloxane and PEG-12 Dimethicone Crosspolymer.

Mention may also be made of the product sold under the name Dow Corning EL-7040 Hydro Elastomer Blend® by the company Dow Corning for the compound which has the INCI name: PEG-12 Dimethicone/PPG-20 Crosspolymer.

b) Polyglycerolated Silicone Elastomers

The polyglycerolated silicone elastomer is an elastomeric crosslinked organopolysiloxane that may be obtained by a crosslinking addition reaction of diorganopolysiloxane containing at least one hydrogen bonded to silicon and of polyglycerolated compounds containing ethylenically unsaturated groups, in particular in the presence of a platinum catalyst.

Preferably, the elastomeric crosslinked organopolysiloxane is obtained by a crosslinking addition reaction (A) of diorganopolysiloxane containing at least two hydrogens each bonded to a silicon, and (B) of glycerolated compounds containing at least two ethylenically unsaturated groups, in particular in the presence (C) of a platinum catalyst.

In particular, the organopolysiloxane may be obtained by reaction of a dimethylvinylsiloxy-terminated polyglycerolated compound and of trimethylsiloxy-terminated methylhydropolysiloxane, in the presence of a platinum catalyst.

Compound (A) is the base reactant for the formation of elastomeric organopolysiloxane, and the crosslinking takes place via an addition reaction of compound (A) with compound (B) in the presence of the catalyst (C).

Compound (A) is in particular an organopolysiloxane having at least two hydrogen atoms bonded to separate silicon atoms in each molecule.

Compound (A) may have any molecular structure, in particular a linear-chain or branched-chain structure or a cyclic structure.

Compound (A) may have a viscosity at 25° C. ranging from 1 to 50 000 centistokes, in particular so as to be readily miscible with compound (B).

The organic groups bonded to the silicon atoms of compound (A) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from methyl, phenyl and lauryl groups.

Compound (A) may thus be chosen from trimethylsiloxy-terminated methylhydropolysiloxanes, trimethylsiloxy-terminated dimethylsiloxane/methylhydrosiloxane copolymers, dimethylsiloxane/methylhydrosiloxane cyclic copolymers, and trimethylsiloxy-terminated dimethylsiloxane/methylhydrosiloxane/laurylmethylsiloxane copolymers.

Compound (B) may be a polyglycerolated compound corresponding to formula (B′) below:

CₘH₂ₘ₋₁—O-[Gly]ₙ-CₘH₂ₘ₋₁ (B′)

in which m is an integer ranging from 2 to 6, n is an integer ranging from 2 to 200, preferably ranging from 2 to 100, preferably ranging from 2 to 50, preferably n ranging from 2 to 20, preferably ranging from 2 to 10 and preferentially ranging from 2 to 5, and in particular n is equal to 3; Gly denotes:

—CH₂—CH(OH)—CH₂—O— or —CH₂—CH(CH₂OH)—O—

Advantageously, the sum of the number of ethylenic groups per molecule in compound (B) and of the number of hydrogen atoms bonded to silicon atoms per molecule in compound (A) is at least 4.

It is advantageous for compound (A) to be added in an amount such that the molecular ratio between the total amount of hydrogen atoms bonded to silicon atoms in compound (A) and the total amount of all the ethylenically unsaturated groups in compound (B) is within the range from 1/1 to 20/1.

Compound (C) is the catalyst for the crosslinking reaction, and is in particular chloroplatinic acid, chloroplatinic acid-olefin complexes, chloroplatinic acid-alkenylsiloxane complexes, chloroplatinic acid-diketone complexes, platinum black and platinum on a support.

Catalyst (C) is preferably added in an amount of from 0.1 to 1000 parts by weight and better still from 1 to 100 parts by weight, as clean platinum metal, per 1000 parts by weight of the total amount of compounds (A) and (B).

The polyglycerolated silicone elastomer according to the invention is generally mixed with at least one hydrocarbon-based oil and/or one silicone oil to form a gel. In these gels, the polyglycerolated elastomer is often in the form of non-spherical particles.

Such elastomers are described in particular in patent application WO 2004/024798.

Use may be made, as polyglycerolated silicone elastomers, of the following compounds having the INCI name:

### Dimethicone/Polyglycerin-3 Crosspolymer,

### Lauryl Dimethicone/Polyglycerin-3 Crosspolymer,

and mixtures thereof.

They are in particular sold by the company Shin Etsu under the following names:

KSG-710®; INCI name: Dimethicone/Polyglycerin-3 Crosspolymer and Dimethicone;
KSG-810®; INCI name: Mineral Oil and Lauryl Dimethicone/Polyglycerin-3 Crosspolymer;
KSG-820®; INCI name: Isododecane and Lauryl Dimethicone/Polyglycerin-3 Crosspolymer;
KSG-830®; INCI name: Triethylhexanoin and Lauryl Dimethicone/Polyglycerin-3 Crosspolymer;
KSG-840®; INCI name: Squalane and Lauryl Dimethicone/Polyglycerin-3 Crosspolymer.

According to one preferred mode, use will be made of the polyoxyalkylenated silicone elastomer Dimethicone/PEG-10/15-Crosspolymer and in particular in the form of a mixture with a dimethicone (INCI name: Dimethicone and Dimethicone/PEG-10/15-Crosspolymer) such as the product sold under the name KSG-210® by the company Shin Etsu.

The emulsifier(s) is (are) present in the composition of the invention preferably in a content of active material of less than 2.5%, more preferentially ranging from 0.1% to 2%, even more preferentially ranging from 0.5% to 1.5% by weight relative to the total weight of said composition.

### Moisturizing Agent

The term “moisturizing agent” is intended to mean, according to the present invention, any compound capable of penetrating into the stratum corneum and of keeping the latter moisturized.

The moisturizing agents that can be used according to the invention are in particular chosen from polyols, urea and its derivatives, such as in particular hydroxyalkyl urea, in particular hydroxyethylurea such as the product sold under the trade name Hydrovance® by the company Akzo Nobel, hyaluronic acid, glycine, β-alanine, taurine, trimethyl glycine, and mixtures thereof.

For the purposes of the present invention, the term “polyol” should be understood as meaning any organic molecule comprising at least two free hydroxyl groups.

According to one particular form, the polyol may be chosen from sugars such as trehalose, mannitol, xylitol, sorbitol, and mixtures thereof.

Preferably, a polyol in accordance with the present invention is present in liquid form at ambient temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those in particular containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols, such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

According to a particular mode, the composition of the invention may comprise at least propylene glycol and/or glycerol.

The moisturizing agent(s) are preferably present in the composition in a content ranging from 10% to 25% by weight, preferably from 15% to 20% by weight, relative to the total weight of said composition.

### Hydrophobic Coated Pigments

The term “pigments” is intended to mean white or coloured, mineral or organic particles, which are insoluble in an aqueous medium, and which are intended to colour and/or opacify the resulting composition and/or film. These pigments may be white or coloured, and mineral and/or organic.

The term “hydrophobic coated pigment” is intended to mean any pigment coated with at least one lipophilic or hydrophobic compound.

The term “lipophilic compound” is intended to mean any compound that is soluble or dispersible in an oily phase.

The term “hydrophobic compound” is intended to mean any compound that is insoluble in water.

According to one particular embodiment, the hydrophobically modified pigments used according to the invention are chosen from mineral pigments.

The term “mineral pigment” is intended to mean any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder or copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The particular size of the coated pigment is strictly greater than 100 nm.

For the purpose of the invention, the term “size” of a particle is intended to mean its D50. The D50, or volume average size, corresponds to the particle size defined such that 50% by volume of the particles have a size less than D50.

The volume average size can be assessed by light diffraction using a Malvern MasterSizer laser particle size analyser, said particles to be evaluated being dispersed in a liquid medium such as, for example, octyldodecyl neopentanoate.

According to one embodiment, the size of the pigment particles according to the invention ranges from 100 nm to 25 μm, preferably from 200 nm to 10 μm.

In the context of the present invention, the hydrophobically modified mineral pigments are more particularly hydrophobically modified pigments of iron oxide and/or titanium dioxide.

They may also be nacres and/or particles with metallic tints.

The term “nacres” should be understood as meaning iridescent or non-iridescent coloured particles of any shape, in particular produced by certain molluscs in their shell or alternatively synthesized, which have a colour effect via optical interference.

The nacres can be chosen from pearlescent pigments, such as titanium oxide-coated mica covered with an iron oxide, titanium oxide-coated mica covered with bismuth oxychloride, titanium oxide-coated mica covered with chromium oxide, titanium oxide-coated mica covered with an organic dye and also pearlescent pigments based on bismuth oxychloride. They can also be mica particles, at the surface of which are superposed at least two successive layers of metal oxides and/or of organic colorants.

Examples of nacres that may also be mentioned include natural mica covered with titanium oxide, with iron oxide, with natural pigment or with bismuth oxychloride.

The nacres may more particularly have a yellow, pink, red, bronze, orange, brown, gold and/or coppery colour or glint.

Advantageously, the nacres in accordance with the invention are micas covered with titanium dioxide or with iron oxide, and also bismuth oxychloride.

For the purposes of the present invention, the term “particles with a metallic tint” is intended to mean any compound whose nature, size, structure and surface finish allow it to reflect the incident light, in particular in a non-iridescent manner.

The particles with a metallic glint which can be used in the invention are chosen in particular from:
particles of at least one metal and/or of at least one metal derivative;
particles comprising a monomaterial or multimaterial, organic or mineral substrate, at least partially coated with at least one layer with a metallic tint comprising at least one metal and/or at least one metal derivative; and
mixtures of said particles.

Mention may be made, among the metals which can be present in said particles, for example, of Ag, Au, Cu, Al, Ni, Sn, Mg, Cr, Mo, Ti, Zr, Pt, Va, Rb, W, Zn, Ge, Te, Se and mixtures thereof or alloys. Ag, Au, Cu, Al, Zn, Ni, Mo, Cr and mixtures thereof or alloys (for example, bronzes and brasses) are preferred metals.

The term “metal derivatives” denotes compounds derived from metals, in particular oxides, fluorides, chlorides and sulfides.

### Coating of the Pigment

The composition according to the invention advantageously comprises at least one pigment coated with at least one lipophilic or hydrophobic compound.

The coating may also comprise at least one additional non-lipophilic compound. For the purposes of the invention, the “coating” of a pigment according to the invention generally denotes the total or partial surface treatment of the pigment with a surface agent, absorbed, adsorbed or grafted onto said pigment.

The surface-treated pigments may be prepared according to surface treatment techniques of chemical, electronic, mechanochemical or mechanical nature that are well known to those skilled in the art. Commercial products may also be used.

The surface agent may be absorbed, adsorbed or grafted onto the pigments by evaporation of solvent, chemical reaction and creation of a covalent bond.

According to one variant, the surface treatment is constituted of a coating of the pigments.

The coating may represent from 0.1% to 20% by weight and in particular from 0.5% to 10% by weight relative to the total weight of the coated pigment.

The coating may be performed, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be performed, for example, by chemical reaction of a surface agent with the surface of the solid pigment particles and creation of a covalent bond between the surface agent and the particles. This method is in particular described in patent U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited at the surface of the pigments.

### Lipophilic or Hydrophobic Treatment Agent

According to a particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

### Silicone Surface Agent

According to a particular embodiment, the pigments may be totally or partially surface-treated with a compound of silicone nature.

The silicone surface agents may be chosen from organopolysiloxanes, silane derivatives, silicone-acrylate copolymers, silicone resins, and mixtures thereof.

The term “organopolysiloxane compound” is intended to mean a compound having a structure comprising an alternance of silicon atoms and oxygen atoms and comprising organic radicals linked to silicon atoms.

### i) Non-Elastomeric Organopolysiloxane

Non-elastomeric organopolysiloxanes that may in particular be mentioned include polydimethylsiloxanes, polymethylhydrogenosiloxanes and polyalkoxydimethylsiloxanes.

The alkoxy group may be represented by the radical R—O— such that R represents methyl, ethyl, propyl, butyl or octyl, 2-phenylethyl, 2-phenylpropyl or 3,3,3-trifluoropropyl radicals, aryl radicals such as phenyl, tolyl or xylyl, or substituted aryl radicals such as phenylethyl.

One method for surface-treating pigments with a polymethylhydrogenosiloxane consists in dispersing the pigments in an organic solvent and then in adding the silicone compound. On heating the mixture, covalent bonds are created between the silicone compound and the surface of the pigment. According to a preferred embodiment, the silicone surface agent may be a non-elastomeric organopolysiloxane, in particular chosen from polydimethylsiloxanes.

According to one particular form, use may be made of triethoxysilylethyl polydimethylsiloxyethyl dimethicone, such as the product sold under the name KF9908® from Shin Etsu.

### ii) Alkylsilanes and Alkoxysilanes

Silanes bearing alkoxy functionality are in particular described by Witucki in “A silane primer, Chemistry and applications of alkoxy silanes, Journal of Coatings Technology, 65, 822, pages 57-60, 1993”.

Alkoxysilanes such as the alkyltriethoxysilanes and the alkyltrimethoxysilanes sold under the references Silquest A-137 (OSI Specialities) and Prosil 9202 (PCR) may be used for coating the pigments.

The use of alkylpolysiloxanes bearing a reactive end group such as alkoxy, hydroxyl, halogen, amino or imino is described in patent application JP H07-196946. They are also suitable for treating the pigments.

iii) Silicone-Acrylate Polymers

Grafted silicone-acrylic polymers having a silicone backbone as described in patents U.S. Pat. No. 5,725,882, U.S. Pat. No. 5,209,924, U.S. Pat. No. 4,972,037, U.S. Pat. No. 4,981,903, U.S. Pat. No. 4,981,902 and U.S. Pat. No. 5,468,477 and in patents U.S. Pat. No. 5,219,560 and EP 0 388 582 may be used.

Other silicone-acrylate polymers may be silicone polymers comprising in their structure the unit of formula (I) below:

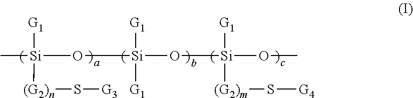

in which the radicals G₁, which may be identical or different, represent hydrogen or a C₁-C₁₀ alkyl radical or alternatively a phenyl radical; the radicals G₂, which may be identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer; m and n are equal to 0 or 1; a is an integer ranging from 0 to 50; b is an integer that may be between 10 and 350, c is an integer ranging from 0 to 50; with the proviso that one of the parameters a and c is other than 0.

Preferably, the unit of formula (I) above has at least one, and even more preferentially all, of the following characteristics:
the radicals G₁ denote an alkyl radical, preferably a methyl radical;
n is non-zero, and the radicals G₂ represent a divalent C₁-C₃ radical, preferably a propylene radical;
G₃ represents a polymeric radical resulting from the (homo)polymerization of at least one monomer of the ethylenically unsaturated carboxylic acid type, preferably acrylic acid and/or methacrylic acid;
G₄ represents a polymeric radical resulting from the (homo)polymerization of at least one monomer of the (C₁-C₁₀)alkyl (meth)acrylate type, preferably such as isobutyl or methyl (meth)acrylate.

Examples of silicone polymers corresponding to formula (I) are in particular polydimethylsiloxanes (PDMS) onto which are grafted, via a connecting chain unit of thiopropylene type, mixed polymer units of the poly(meth)acrylic acid type and of the polymethyl (meth)acrylate type.

Other examples of silicone polymers corresponding to formula (I) are in particular polydimethylsiloxanes (PDMS) onto which are grafted, via a connecting chain unit of thiopropylene type, polymer units of the polyisobutyl (meth)acrylate type.

### iv) Silicone Resins

The silicone surface agent may be chosen from silicone resins.

The term “resin” is intended to mean a three-dimensional structure.

The silicone resins may be soluble or swellable in silicone oils. These resins are crosslinked polyorganosiloxane polymers.

The nomenclature of silicone resins is known under the name “MDTQ”, the resin being described as a function of the various siloxane monomer units that it comprises, each of the letters “MDTQ” characterizing a type of unit.

The letter M represents the monofunctional unit of formula (CH₃)₃SiO_{1/2}, the silicon atom being connected to only one oxygen atom in the polymer comprising this unit.

The letter D signifies a difunctional unit (CH₃)₂SiO_{2/2} in which the silicon atom is bonded to two oxygen atoms.

The letter T represents a trifunctional unit of formula (CH₃)SiO_{3/2}.

In the units M, D and T defined above, at least one of the methyl groups may be substituted with a group R other than a methyl group, such as a hydrocarbon-based radical (in particular alkyl) containing from 2 to 10 carbon atoms or a phenyl group, or alternatively a hydroxyl group.

Finally, the letter Q means a tetrafunctional unit SiO_{4/2} in which the silicon atom is bonded to four hydrogen atoms, which are themselves bonded to the rest of the polymer.

Various resins with different properties may be obtained from these different units, the properties of these polymers varying as a function of the type of monomers (or units), of the type and number of substituted radicals, of the length of the polymer chain, of the degree of branching and of the size of the pendent chains.

Examples of these silicone resins that may be mentioned include:
siloxysilicates, which may be trimethyl siloxysilicates of formula [(CH₃)₃XSiXO]ₓX(SiO_{4/2})_{y} (MQ units) in which x and y are integers ranging from 50 to 80;
   polysilsesquioxanes of formula (CH₃SiO_{3/2})ₓ (T units) in which x is greater than 100 and at least one of the methyl radicals of which may be substituted with a group R as defined above;
polymethylsilsesquioxanes, which are polysilsesquioxanes in which none of the methyl radicals is substituted with another group. Such polymethylsilsesquioxanes are described in document U.S. Pat. No. 5,246,694.

As examples of commercially available polymethylsilsesquioxane resins, mention may be made of those sold:
by the company Wacker under the reference Resin MK®, such as Belsil PMS MK®: polymer comprising CH₃SiO_{3/2} repeating units (T units), which may also comprise up to 1% by weight of (CH₃)₂SiO_{2/2} units (D units) and having an average molecular weight of about 10 000;
by the company Shin-Etsu under the references KR-220L®, which are composed of T units of formula CH₃SiO_{3/2} and contain Si—OH (silanol) end groups, under the reference KR-242A, which comprise 98% of T units and 2% of dimethyl D units and contain Si—OH end groups, or also under the reference KR-251, comprising 88% of T units and 12% of dimethyl D units and containing Si—OH end groups.

Siloxysilicate resins that may be mentioned include trimethyl siloxysilicate (TMS) resins, optionally in the form of powders. Such resins are sold under the references SR10000, E 1 170-002® or SS 4230®, by the company General Electric or under the references TMS 803®, Wacker 803® and 804® by the company Wacker Silicone Corporation.

Mention may also be made of trimethylsiloxysilicate resins sold in a solvent such as cyclomethicone, sold under the name KF-7312J® by the company Shin-Etsu or DC 749® and DC 593® by the company Dow Corning.

As examples of commercial references of pigments treated with a silicone compound, mention may be made of:
red iron oxide/dimethicone sold under the reference SA-C 338075-10® by the company Miyoshi Kasei; and

### Fluoro Surface Agent

The pigments may be totally or partially surface-treated with a compound of fluoro nature.

The fluoro surface agents may be chosen from perfluoroalkyl phosphates, perfluoropolyethers, polytetrafluoropolyethylenes (PTFE), perfluoroalkanes, perfluoroalkyl silazanes, polyhexafluoropropylene oxides, and polyorganosiloxanes comprising perfluoroalkyl perfluoropolyether groups.

The term “perfluoroalkyl radical” is intended to mean an alkyl radical in which all of the hydrogen atoms have been replaced with fluorine atoms.

Perfluoropolyethers are in particular described in patent application EP 0 486 135, and sold under the trade name Fomblin® by the company Montefluos.

Perfluoroalkyl phosphates are described in particular in patent application JP H05-86984. The perfluoroalkyl diethanolamine phosphates sold by Asahi Glass under the reference AsahiGuard AG530® may be used.

Among the linear perfluoroalkanes that may be mentioned are perfluorocycloalkanes, perfluoro(alkylcycloalkanes), perfluoropolycycloalkanes, aromatic perfluoro hydrocarbons (perfluoroarenes) and hydrocarbon-based perfluoro organic compounds comprising at least one heteroatom. Among the perfluoroalkanes, mention may be made of the linear alkane series such as perfluorooctane, perfluorononane or perfluorodecane.

Among the perfluorocycloalkanes and perfluoro(alkylcycloalkanes), mention may be made of perfluorodecalin sold under the name Flutec PP5 GMP® by the company Rhodia, perfluoro(methyldecalin) and perfluoro(C₃-C₅ alkylcyclohexanes) such as perfluoro(butylcyclohexane).

Among the perfluoropolycycloalkanes, mention may be made of bicyclo[3.3.1]nonane derivatives such as perfluorotrimethylbicyclo[3.3.1]nonane, adamantane derivatives such as perfluorodimethyladamantane, and hydrogenated perfluorophenanthrene derivatives such as tetracosafluorotetradecahydrophenanthrene.

Among the perfluoroarenes, mention may be made of perfluoronaphthalene derivatives, for instance perfluoro-naphthalene and perfluoromethyl-1-naphthalene.

As examples of commercial references of pigments treated with a fluoro compound, mention may be made of:
yellow iron oxide/perfluoroalkyl phosphate sold under the reference PF 5 Yellow 601® by the company Daito Kasei;
red iron oxide/perfluoroalkyl phosphate sold under the reference PF 5 Red R 516L® by the company Daito Kasei;
black iron oxide/perfluoroalkyl phosphate sold under the reference PF 5 Black BL100® by the company Daito Kasei;
titanium dioxide/perfluoroalkyl phosphate sold under the reference PF 5 TiO₂ CR 50® by the company Daito Kasei;
yellow iron oxide/perfluoropolymethyl isopropyl ether sold under the reference Iron oxide yellow BF-25-3® by the company Toshiki;
DC Red 7/perfluoropolymethyl isopropyl ether sold under the reference D&C Red 7 FHC® by the company Cardre Inc.; and
DC Red 6/PTFE sold under the reference T 9506® by the company Warner-Jenkinson.

### Fluorosilicone Surface Agent

The pigments may be totally or partially surface-treated with a compound of fluorosilicone nature.

The fluorosilicone compound may be chosen from perfluoroalkyl dimethicones, perfluoroalkyl silanes and perfluoroalkyl trialkoxysilanes.

Perfluoroalkyl silanes that may be mentioned include the products LP-IT® and LP-4T® sold by Shin-Etsu Silicone.

The perfluoroalkyl dimethicones may be represented by the following formula:

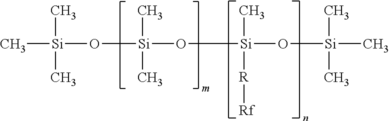

in which:
R represents a linear or branched divalent alkyl group containing from 1 to 6 carbon atoms, preferably a divalent methyl, ethyl, propyl or butyl group;
Rf represents a perfluoroalkyl radical containing 1 to 9 carbon atoms and preferably 1 to 4 carbon atoms;
m is chosen between 0 and 150 and preferably from 20 to 100; and
n is chosen between 1 and 300 and preferably from 1 to 100.

As examples of commercial references of pigments treated with a fluorosilicone compound, mention may be made of titanium dioxide/fluorosilicone sold under the reference Fluorosil Titanium dioxide 100TA® by the company Advanced Dermaceuticals International Inc.

### Other Lipophilic Surface Agents

The hydrophobic treatment agent may also be chosen from:

i) metal soaps such as aluminium dimyristate and the aluminium salt of hydrogenated tallow glutamate;

Metal soaps that may in particular be mentioned include metal soaps of fatty acids containing from 12 to 22 carbon atoms and in particular those containing from 12 to 18 carbon atoms.

The metal of the metal soap may in particular be zinc or magnesium.

Metal soaps that may be used include zinc laurate, magnesium stearate, magnesium myristate and zinc stearate, and mixtures thereof;

ii) fatty acids such as lauric acid, myristic acid, stearic acid and palmitic acid;
iii) N-acylamino acids or salts thereof, which may comprise an acyl group containing from 8 to 22 carbon atoms, for instance a 2-ethylhexanoyl, caproyl, lauroyl, myristoyl, palmitoyl, stearoyl or cocoyl group.

The amino acid may be, for example, lysine, glutamic acid or alanine.

The salts of these compounds may be the aluminium, magnesium, calcium, zirconium, zinc, sodium or potassium salts.

Thus, according to a particularly preferred embodiment, an N-acylamino acid derivative may in particular be a glutamic acid derivative and/or a salt thereof, and more particularly a stearoyl glutamate, for instance aluminium stearoyl glutamate. It is, for example, the NAI surface treatments sold by Miyoshi;

iv) lecithin and derivatives thereof, such as hydrogenated lecithin, for instance the HLC surface treatment sold by LCW;
v) isopropyl triisostearyl titanate (INCI name: Isopropyl Titanium Triisostearate).

As examples of isopropyl titanium triisostearate (ITT)-treated pigments, mention may be made of those sold under the commercial references BTD-401® (titanium dioxide CI77891 and isopropyl titanium triisostearate), BBO-I2® (iron oxide CI77499 and isopropyl titanium triisostearate), BYO-I2® (iron oxide CI77492 and isopropyl titanium triisostearate) and BRO-I2® (iron oxide CI77491 and isopropyl titanium triisostearate) by the company Kobo;

vi) isostearyl sebacate;
vii) natural plant or animal waxes or polar synthetic waxes;
viii) fatty esters, in particular jojoba esters;
ix) phospholipids; and
x) mixtures thereof.

The waxes mentioned in the compounds mentioned previously may be those generally used in cosmetics, as defined hereinbelow.

They may in particular be hydrocarbon, silicone and/or fluoro waxes, optionally comprising ester or hydroxyl functions. They may also be of natural or synthetic origin.

The term “polar wax” is intended to mean a wax containing chemical compounds comprising at least one polar group. Polar groups are well known to those skilled in the art; they may be, for example, alcohol, ester or carboxylic acid groups. Polyethylene waxes, paraffin waxes, microcrystalline waxes, ozokerite and Fischer-Tropsch waxes are not included among polar waxes.

In particular, the polar waxes have a mean Hansen solubility parameter δₐ at 25° C. such that δₐ>0 (J/cm³)^{1/2} and better still δₐ>1 (J/cm³)^{1/2}:

δₐ=\sqrt{δₚ²+δₕ²}

in which δₚ and δₕ are, respectively, the polar contributions and contributions of interaction types specific to the Hansen solubility parameters.

The definition of solvents in the three-dimensional solubility space according to Hansen is described in the article by C. M. Hansen: “The three-dimensional solubility parameters”, J. Paint Technol. 39, 105 (1967):
δₕ characterizes the specific interaction forces (such as hydrogen bonding, acid/base, donor/acceptor, etc.);
δₚ characterizes the Debye interaction forces between permanent dipoles and also the Keesom interaction forces between induced dipoles and permanent dipoles.

The solubility parameters are calculated with the HSPiP v4.1 software.

The parameters δₚ and δₕ are expressed in (J/cm³)^{1/2}.

A polar wax is in particular formed from molecules comprising, besides carbon and hydrogen atoms in their chemical structure, heteroatoms (such as O, N and P).

Non-limiting illustrations of these polar waxes that may in particular be mentioned include natural polar waxes, such as beeswax, lanolin wax, orange wax, lemon wax and Chinese insect waxes, rice bran wax, carnauba wax, candelilla wax, ouricury wax, cork fibre wax, sugarcane wax, Japan wax, sumac wax and montan wax.

According to one particular embodiment, the pigments may be coated with at least one compound chosen from N-acylamino acids or salts thereof, isopropyl triisostearyl titanate; silicone surface agents; natural plant or animal waxes; hydrogenated lecithin, fatty esters; and mixtures thereof.

According to a more particularly preferred embodiment, the pigments may be coated with an N-acylamino acid and/or a salt thereof, in particular with a glutamic acid derivative and/or a salt thereof, in particular a stearoyl glutamate, for instance aluminium stearoyl glutamate.

According to one more particularly preferred embodiment, use will be made of hydrophobic coated pigments chosen from titanium dioxides and iron oxides coated with aluminium stearoyl glutamate, sold, for example, under the reference NAI® by Miyoshi Kasei.

According to a more particularly preferred embodiment, use will be made of hydrophobic coated pigments chosen from titanium dioxides and iron oxides coated with isopropyl titanium triisostearate (ITT); mention may be made of those sold under the commercial references BTD-401® (titanium dioxide CI77891 and isopropyl titanium triisostearate), BBO-I2® (iron oxide CI77499 and isopropyl titanium triisostearate), BYO-I2® (iron oxide CI77492 and isopropyl titanium triisostearate), and BRO-I2® (iron oxide CI77491 and isopropyl titanium triisostearate) by the company Kobo.

The hydrophobic coated pigments are present in a composition of the invention, preferably, in a proportion of at least 5% by weight, more preferentially ranging from 5% to 25% by weight, more preferentially ranging from 8% to 15% by weight relative to the total weight of the composition.

According to a more particularly preferred embodiment, the water-in-oil emulsion of the invention comprises:
an aqueous phase at a concentration ranging from 60% to 80% by weight, more particularly from 65% to 75% by weight, relative to the total weight of said composition; and
at least one moisturizing agent at a concentration ranging from 5% to 30%, better still from 10% to 25% by weight, preferably from 15% to 20% by weight, relative to the total weight of said composition; and
at least one hydrophobic coated pigment in a concentration of at least 5% by weight, more preferentially ranging from 5% to 25% by weight, even more preferentially ranging from 8% to 15% by weight relative to the total weight of the composition;
   said composition having a viscosity at 25° C. ranging from 0.5 to 5 Pa·s, more preferentially from 1.5 to 3 Pa·s.

### Additional Colorants

A composition according to the invention may also comprise at least one additional colorant, preferably in a proportion of at least 0.01% by weight relative to the total weight of the composition.

For obvious reasons, this amount is liable to vary significantly with regard to the intensity of the desired colour effect and of the colour intensity afforded by the colorants under consideration, and its adjustment clearly falls within the competence of those skilled in the art.

A composition according to the invention may comprise from 0.01% to 25% by weight, in particular from 0.1% to 25% by weight, in particular from 1% to 20% by weight and preferably from 5% to 15% by weight of colorants relative to the total weight of said composition.

As specified above, the colorants that are suitable for use in the invention may be water-soluble, but may also be liposoluble.

For the purposes of the invention, the term “water-soluble colorant” is intended to mean any natural or synthetic, generally organic compound, which is soluble in an aqueous phase or water-miscible solvents and which is capable of imparting colour.

As water-soluble dyes that are suitable for use in the invention, mention may be made in particular of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophylline, methylene blue, anthocyanins (enocianin, black carrot, hibiscus and elder), caramel and riboflavin.

The water-soluble dyes are, for example, beetroot juice and caramel.

For the purposes of the invention, the term “liposoluble colorant” is intended to mean any natural or synthetic, generally organic compound, which is soluble in an oily phase or in solvents that are miscible with a fatty substance, and which is capable of imparting colour.

As liposoluble dyes that are suitable for use in the invention, mention may be made in particular of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

They may in particular be non-hydrophobic-coated pigments, non-hydrophobic-coated nacres and/or non-hydrophobic-coated particles with metallic tints.

The term “non-hydrophobic-coated pigment” is intended to mean any pigment which is not coated with at least one lipophilic or hydrophobic compound.

The additional pigments may be white or coloured, and mineral and/or organic.

As non-hydrophobic-coated mineral pigments that may be used in the invention, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

They may also be a pigment having a structure that may be, for example, of sericite/brown iron oxide/titanium dioxide/silica type. Such a pigment is sold, for example, under the reference Coverleaf NS® or JS® by the company Chemicals and Catalysts, and has a contrast ratio in the region of 30.

They may also be pigments having a structure that may be, for example, of silica microsphere type containing iron oxide. An example of a pigment having this structure is the product sold by the company Miyoshi under the reference PC Ball PC-LL-100 P®, this pigment being constituted of silica microspheres containing yellow iron oxide.

Advantageously, the additional pigments in accordance with the invention are iron oxides and/or titanium dioxides.

The nacres can be chosen from pearlescent pigments, such as titanium oxide-coated mica covered with an iron oxide, titanium oxide-coated mica covered with bismuth oxychloride, titanium oxide-coated mica covered with chromium oxide, titanium oxide-coated mica covered with an organic dye and also pearlescent pigments based on bismuth oxychloride. They can also be mica particles, at the surface of which are superposed at least two successive layers of metal oxides and/or of organic colorants.

Examples of nacres that may also be mentioned include natural mica covered with titanium oxide, with iron oxide, with natural pigment or with bismuth oxychloride.

Among the nacres available on the market, mention may be made of the nacres Timica, Flamenco® and Duochrome® (based on mica) sold by the company Engelhard, the Timiron nacres sold by the company Merck, the Prestige mica-based nacres, sold by the company Eckart, and the Sunshine synthetic mica-based nacres, sold by the company Sun Chemical.

The nacres may more particularly have a yellow, pink, red, bronze, orange, brown, gold and/or coppery colour or glint.

Advantageously, the nacres in accordance with the invention are micas covered with titanium dioxide or with iron oxide, and also bismuth oxychloride.

Illustrations of these additional particles with metallic tints that may be mentioned include aluminium particles, such as those sold under the names Starbrite 1200 EAC® by the company Siberline and Metalure® by the company Eckart and glass particles coated with a metallic layer, in particular those described in documents JP-A-09188830, JP-A-10158450, JP-A-10158541, JP-A-07258460 and JP-A-05017710.

### Fillers

Advantageously, a composition according to the invention may also comprise one or more fillers conventionally used in care and/or makeup compositions.

These fillers are colourless or white solid particles of any form, which are in a form that is insoluble and dispersed in the medium of the composition.

These fillers, of mineral or organic, natural or synthetic nature, give the composition containing them softness and give the makeup result a matt effect and uniformity. In addition, these fillers advantageously make it possible to combat various attacking factors such as sebum or sweat.

As illustrations of these fillers, mention may be made of talc, mica, silica, kaolin, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), acrylic acid copolymer microspheres, silicone resin microbeads (for example Tospearls® from Toshiba), polyorganosiloxane elastomer particles, precipitated calcium carbonate, magnesium carbonate, magnesium hydrogen carbonate, hydroxyapatite, barium sulfate, aluminium oxides, polyurethane powders, composite fillers, hollow silica microspheres, and glass or ceramic microcapsules. Use may also be made of particles which are in the form of hollow sphere portions, as described in patent applications JP-2003 128 788 and JP-2000 191 789.

In particular, such fillers may be present in a composition according to the invention in a content ranging from 0.01% to 25% by weight, in particular from 0.1% to 20% by weight, in particular from 1% to 10% by weight, relative to the total weight of the composition.

### Active Agent

Mention may be made, as active agents which can be used in the composition of the invention, for example, of vitamins, sunscreens other than the liquid lipophilic organic UV-screening agents, and mixtures thereof.

Preferably, a composition according to the invention comprises at least one active agent.

It is a matter of routine operations for those skilled in the art to adjust the natures and the amounts of the additives present in the compositions in accordance with the invention so that the cosmetic properties desired for the latter are not affected thereby.

### Compositions

According to one particularly preferred form, the compositions according to the invention are in the form of a water-in-oil emulsion.

According to one embodiment, a composition of the invention may advantageously be in the form of a composition for caring for the skin and/or keratin fibres, the body or the face, in particular the face.

According to another embodiment, a composition of the invention can advantageously be in the form of a composition for making up keratin materials, in particular the skin of the body or of the face, in particular of the face.

Thus, according to a sub-mode of this embodiment, a composition of the invention may advantageously be in the form of a makeup base composition.

A composition of the invention may advantageously be in the form of a foundation.

According to another sub-mode of this embodiment, a composition of the invention may advantageously be in the form of a composition for making up the skin and in particular the face. It may thus be an eyeshadow or a face powder.

According to yet another sub-mode of this embodiment, a composition of the invention may advantageously be in the form of a product for making up the lips, in particular a lipstick.

According to yet another sub-mode of this embodiment, a composition of the invention may advantageously be in the form of a product for making up and/or caring for the eyebrows.

Such compositions are prepared in particular according to the general knowledge of those skilled in the art.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

The invention is illustrated in more detail by the examples and figures presented below. Unless otherwise indicated, the amounts shown are expressed as percentages by weight.

### EXAMPLE

### Examples 1 and 2: W/O Emulsion Foundations

| Phases | INCI names | Ex 1 | Ex 2* |
|---|---|---|---|
| A1 | CAPRYLYL METHICONE | 12.0 | 0 |
| | (DOW CORNING FZ-3196 ®) | | |
| | MINERAL OIL | 0 | 12.0 |
| A2 | DIMETHICONE (and) DIMETHICONE/PEG-10/15 | 4.0 | 4.0 |
| | CROSSPOLYMER | | |
| | (KSG 210 ® from SHIN ETSU) | | |
| | ETHYLHEXYL METHOXYCINNAMATE | 3.0 | 3.0 |
| | (PARSOL MCX ®) | | |
| | FRAGRANCE | 0.2 | 0.2 |
| B | WATER | q.s. for | q.s. for |
| | | 100 | 100 |
| | SODIUM CHLORIDE | 0.5 | 0.5 |
| | GLYCEROL | 15.0 | 15.0 |
| | DISODIUM EDTA | 0.1 | 0.1 |
| | PROPANEDIOL | 3.0 | 3.0 |
| | CHLORPHENESIN | 0.3 | 0.3 |
| | PHENOXYETHANOL | 0.5 | 0.5 |
| C | IRON OXIDES (and) ISOPROPYL TITANIUM | 0.1 | 0.1 |
| | TRIISOSTEARATE (BBO-I2 ® from KOBO) | | |
| | IRON OXIDES (and) ISOPROPYL TITANIUM | 0.3 | 0.3 |
| | TRIISOSTEARATE | | |
| | (BRO-I2 ® from KOBO) | | |
| | IRON OXIDES (and) ISOPROPYL TITANIUM | 0.66 | 0.66 |
| | TRIISOSTEARATE (BYO-I2 ® from KOBO) | | |
| | TITANIUM DIOXIDE (and) ISOPROPYL | 9.0 | 9.0 |
| | TITANIUM TRIISOSTEARATE | | |
| | (BTD-401 ® from KOBO) | | |

*Outside the invention

### Procedure:

The water was heated to 50° C. and all the elements of phase B were incorporated with electromagnetic stirring for 5 minutes; the phase became translucent.

B was left to cool. The pigments were milled in phase A1 using a three-roll mill with 3 openings (large, medium then small). When the milled material was ready, it was introduced into a beaker with A2. The mixture was homogenized in a rotor stator (Moritz) for 15 minutes at 3000 revolutions/minute. The silicone elastomer had to be well dispersed so as to no longer form clumps. A was then stirred in a Rayneri deflocculating paddle mixer at 500 revolutions/minute. B was added over the course of approximately 1 minute while increasing the stirring to 1000 revolutions/minute. The walls were scraped and stirring was continued for a further 2 minutes at 1000 revolutions/minute. The stirring was stopped.

### Comparative Tests of Stability

The study of the stability of a formula consisted in observing its behaviour in the time and its stability in various temperatures. A formula was considered as stable when its texture was homogeneous during the time in various temperatures.

Compositions 1 and 2 were conditioned in 30 ml glass flasks and were stored
1) during 3 days at room temperature (25° C.) and
2) during 15 days in an oven with the following temperature cycles: +20° C. during 6 hours; 6 hours to reach −20° C.; −20° C. during 6 hours and finally 6 hours to reach +20° C.

The macroscopic aspect of each composition 1 and 2, stored in the oven with temperature cycles, was compared with the macroscopic aspect of the corresponding composition at room temperature as control.

The results are indicated in the following table:

| | Ex 1 | Ex 2 |
|---|---|---|
| | (invention) | (outside the |
| | Silicone | invention) |
| Stability | oil | Mineral oil |
| After 3 days in the oven with | Stable | Unstable |
| temperature cycles | | |
| After 15 days at room temperature | Stable | Unstable |
| (control) | | |

Contrarily to the composition 2 containing a non-silicone oil (mineral oil), the composition 1 containing a silicone oil was stable after 3 days at room temperature and after 15 days in the oven with temperature cycles. Furthermore, the composition 1 made it possible to give good makeup and coverage results and also good moisturizing properties.

1. A composition, in the form of an emulsion, comprising a physiologically acceptable medium, comprising:
at least one aqueous phase in a content from 60% to 80% by weight relative to the total weight of the composition;
at least one oily phase at a concentration of less than 30.0% by weight relative to the total weight of the composition and comprising at least one silicone oil;
at least one hydrophobic coated pigment;
optionally at least one moisturizing agent at a concentration ranging from 10% to 25% by weight relative to the total weight of the composition; and
at least one emulsifying surfactant.

2. The composition according to claim 1, comprising:
at least one aqueous phase in a content ranging from 60% to 80% by weight relative to the total weight of the composition;
at least one oily phase, at a concentration of less than 30.0% by weight relative to the total weight of the composition and comprising at least one silicone oil;
at least one hydrophobic coated pigment; and
at least one emulsifying silicone elastomer.

3. The composition according to claim 1, comprising:
at least one aqueous phase in a content from 60% to 80% by weight relative to the total weight of the composition;
at least one continuous oily phase at a concentration of less than 30.0% by weight relative to the total weight of the composition and comprising at least one silicone oil;
at least one hydrophobic coated pigment;
at least one moisturizing agent at a concentration ranging from 10% to 25% by weight relative to the total weight of the composition; and
at least one emulsifying surfactant,
said composition having a viscosity at 25° C. ranging from 0.1 to 10 Pa·s.

4. The composition according to claim 3, wherein the viscosity ranges from 0.5 to 5 Pa·s.

5. The composition according to claim 1, wherein the aqueous phase of the emulsion is characterized by a number of droplets of less than 200,000.

6. The composition according to claim 1, wherein the surfactant emulsifier is chosen from polyoxyalkylenated silicone elastomers and polyglycerolated silicone elastomers, and mixtures thereof.

7. The composition according to claim 1, wherein the emulsifying surfactant is present in a content of active material of less than 2.5% by weight relative to the total weight of said composition.

8. The composition according to claim 1, wherein the oily phase concentration is less than or equal to 25% by weight, relative to the total weight of the composition.

9. The composition according to claim 1, wherein the oily phase comprises at least one silicone oil chosen from:
volatile cyclic silicone oils having a viscosity at ambient temperature of less than 8 cst and containing from 4 to 7 silicone atoms, these silicones optionally comprising alkyl or alkoxy groups containing from 1 to 10 carbon atoms;
volatile or non-volatile polydimethylsiloxanes (PDMSs);
phenylated silicones;
polydimethylsiloxanes comprising aliphatic groups, which are pendent and/or at the end of the silicone chain, these groups each comprising from 6 to 24 carbon atoms, and mixtures thereof.

10. The composition according to claim 1, wherein the oily phase comprises in addition at least one liquid lipophilic organic UV-screening agent, chosen from:
liquid lipophilic β,β-diphenylacrylate compounds,
liquid lipophilic salicylate compounds,
liquid lipophilic cinnamate compounds,
and mixtures thereof.

11. The composition according to claim 1, wherein the oily phase comprises at least ethylhexyl methoxycinnamate and caprylyl methicone.

12. The composition according to claim 1, wherein the moisturizing agent is chosen from polyols, urea and its derivatives, hyaluronic acid, glycine, β-alanine, taurine, trimethyl glycine, and mixtures thereof.

13. The composition according to claim 1, wherein the moisturizing agent is chosen from polyols chosen from ethylene glycol, pentaerythritol, trimethylolpropane, propylene glycol, 1,3 propanediol, butylene glycol, isoprene glycol, pentylene glycol, hexylene glycol, glycerol, polyglycerols, polyethylene glycols, and mixtures thereof.

14. The composition according to claim 1, wherein the moisturizing agent is present in the composition in a content ranging from 15% to 20% by weight relative to the total weight of said composition.

15. The composition according to claim 1, wherein the hydrophobic coated pigments are present in a proportion ranging from 5% to 25% by weight, relative to the total weight of the composition.

16. The composition according to claim 1, wherein the hydrophobic coated pigments are hydrophobic coated pigments of iron oxide and/or of titanium dioxide.

17. The composition according to claim 1, wherein the hydrophobic coated pigments are titanium dioxides and/or iron oxides coated:
with an N-acylamino acid and/or a salt thereof; or
with isopropyl titanium triisostearate.

18. A process for coating keratin materials, comprising applying the composition according to claim 1 to the keratin materials.

19. A process for preparing a water-in-oil emulsion, comprising:
at least one aqueous phase in a content from 60% to 80% by weight relative to the total weight of the composition;
at least one oily phase at a concentration of less than 30.0% by weight relative to the total weight of the composition and comprising at least one silicone oil;
at least one hydrophobic coated pigment;
optionally at least one moisturizing agent at a concentration ranging from 10% to 25% by weight relative to the total weight of the composition;
at least one surfactant emulsifier,
the method comprising:
a) preparing an aqueous phase by mixing the water, the optional moisturizing agent and the optional other ingredients, with magnetic stirring, at a temperature of from 50 to 60° C. from 3 to 10 minutes and then leaving to cool to 25-30° C.;
b) preparing the oily phase, at a temperature ranging from 25 to 30° C., by mixing the hydrophobic coated pigment, the surfactant emulsifier and the oily component with rotor stator stirring (Moritz) for 15 to 25 minutes at a shear rate ranging from 2500 to 3500 revolutions/minute; then
c) stirring the oily phase is stirred in a Rayneri deflocculating paddle mixer at a shear rate ranging from 300 to 500 revolutions/minute, at a temperature of from 25 to 30° C.; then
d) adding the aqueous phase to said oily phase, over the course of a period of time not exceeding 1 minute, while increasing the stirring to a shear rate ranging from 700 to 1200 revolutions/minute, at a temperature of from 25 to 30° C.; and
e) leaving the resulting mixture to stir for a further period of time ranging from 1 to 3 minutes, at a shear rate ranging from 700 to 1200 revolutions/minute, at a temperature ranging from 25 to 30° C.
